(19) United States
(12) Patent Application Publication
(10) Pub. No.: US 2026/0131087 A1
(43) Pub. Date: May 14, 2026

(54) SAFETY SYRINGE SYSTEMS, SAFETY SYRINGES, AND METHODS OF USE

(71) Applicant: LOVE LIFESCIENCES LLC, Olathe, KS (US)

(72) Inventors: Nicholas Love, Olathe, KS (US); Bradley Hopper, Topeka, KS (US)

(21) Appl. No.: 19/118,601

(22) PCT Filed: Oct. 4, 2023
(86) PCT No.: PCT/US23/75922

§ 371 Date: Apr. 4, 2025

Related U.S. Application Data
(60) Provisional application No. 63/378,664, filed on Oct. 6, 2022.

(51) Int. Cl. A61M 5/32; A61M 5/20; A61M 5/315

(52) U.S. Cl. CPC A61M 5/3204; A61M 5/20; A61M 5/3158; A61M 2005/206; A61M 2005/2073; A61M 2005/31588; A61M 2205/502; A61M 2205/8206

(57) ABSTRACT

A syringe safety system including a dispensing device and at least one safety syringe housing a syringe. The dispensing device includes a casing, a cap having a display screen, a microcontroller in communication with the display screen, and a carrier assembly. The carrier assembly configured to be positioned within an inner volume of the casing. The carrier assembly configured to support the at least one safety syringe around a central axis, rotate the at least one injector about the central axis, and dispense the at least one safety syringe out the distal opening in the casing for use.

### CROSS-REFERENCE TO RELATED APPLICATIONS

This application claims the benefit of U.S. Provisional Ser. No. 63/378,664, filed Oct. 6, 2022. This application is hereby incorporated by reference in its entirety into the present application for all purposes.

### FIELD OF THE INVENTION

The present disclosure relates to safety syringe systems, safety syringes, and methods of use. More particularly, the disclosure relates to safety syringe systems with time-controlled release of pre-filled safety syringes that can be manually actuated to deliver a dose of a drug.

### BACKGROUND OF THE INVENTION

Auto-injectors are medical devices that deliver a single dose of a drug via an enclosed, spring-loaded syringe. Conventionally, auto-injectors are designed to be self-administered, but can be administered by others. Auto-injectors are relatively easy to administer as compared with a syringe and needle, which require dosing of the drug into the syringe. Auto-injectors can mitigate fears associated with seeing an exposed needle. But, some patients may also fear the abrupt stab of the needle from auto-injectors.

Despite auto-injectors and conventional syringes and needles, many patients still fear needle injections. The fear can be so great that some patients may even quit administering their medications due to problems they face with auto-injectors and other injection methods. This causes problems for physicians looking to maintain patients on injectable therapies, insurance companies covering patients requiring injectable therapies, and pharmaceutical companies looking to maintain patients on their medications.

In addition to the fear of needle injections, there are challenges associated with maintaining a regular schedule of drug delivery when multiple injections are required over a given timeframe. It is with these thoughts in mind, among others, that aspects of the safety syringe devices for dispensing pre-filled safety syringes, systems, and methods of use were conceived. By utilizing a safety syringe device having multiple pre-filled safety syringes contained therein, patients will be more likely to maintain use of their medications on a regular schedule with less fear of the needle injection.

### SUMMARY OF THE INVENTION

Aspects of the present disclosure may include a syringe safety system including at least one safety syringe and a dispensing device. The at least one safety syringe includes a first safety syringe including a first outer casing configured to enclose a first syringe having a first barrel, a first needle extending distally from the first barrel, and a first plunger insertable within the first barrel to dispense a first drug within the first barrel. The dispensing device includes an outer casing, a cap, a microcontroller, and a carrier assembly. The outer casing includes a sidewall defining an inner volume, a distal opening, and a proximal opening opposite the distal opening. The a cap is configured to couple to and enclose the proximal opening of the outer casing. The microcontroller is configured to be housed within the outer casing and in communication with at least one battery. The carrier assembly is configured to be positioned within the inner volume of the outer casing.

The carrier assembly includes: a carrier clip configured to support the at least one safety syringe around a central axis; a guide tube coupled to the carrier clip and including a first track for guiding linear movement of the first safety syringe; a lumen extending through the guide tube; a lead screw positioned within the lumen; a guide tube motor rotatably coupled to the lead screw and in communication with the microcontroller; a carrier motor rotatably coupled to the guide tube and in communication with the microcontroller; a lead nut engaged with the lead screw and including a protrusion that is sized and shaped to be received in the first track to guide linear movement of the first safety syringe; and a first plunger coupler including a first plunger support portion and a first protrusion extending outwards from the first plunger support portion. The first protrusion is configured to be received within the first track. The first plunger support portion is positioned to engage the first plunger of the first syringe. The protrusion of the lead nut is configured to contact the first protrusion of the first plunger coupler in the first track such that linear movement in a distal direction of the lead nut via rotation of the lead screw by the guide tube motor is configured to cause distal advancement of the first plunger support portion and the first safety syringe at least partially out of the distal opening of the outer casing.

In certain instances, the at least one safety syringe further includes a second safety syringe including a second outer casing configured to enclose a second syringe having a second barrel, a second needle extending distally from the second barrel, and a second plunger insertable within the second barrel to dispense a second drug within the second barrel. The guide tube further includes a second track for guiding linear movement of the second safety syringe, the protrusion of the lead nut being sized and shaped to be received in the second track to guide linear movement of the second safety syringe. The carrier assembly further includes a second plunger coupler including a second plunger support portion and a second protrusion extending outwards from the second plunger support portion, the second protrusion configured to be received within the second track, the second plunger support portion positioned to engage the second plunger of the second syringe. Wherein the lead nut is configured to be positioned proximally of the second protrusion in the second track such that linear movement in a distal direction of the lead nut via rotation of the lead screw by the guide tube motor is configured to cause distal advancement of the second plunger coupler and the second safety syringe at least partially out of the distal opening of the outer casing.

In certain instances, the microcontroller is programmable to permit actuation of the lead screw in order to distally advance the second safety syringe at least partially out of the distal opening of the outer casing for dispensing of the second drug at a predetermined amount of time after the first safety syringe is distally advanced at least partially out of the distal opening of the outer casing.

In certain instances, subsequent to distal advancement of the first safety syringe at least partially out of the distal opening of the outer casing, the microcontroller is configured to: proximally retract the first safety syringe into the inner volume of the outer casing via rotation of the lead screw by the guide tube motor; and rotate the carrier assembly within the inner volume of the outer casing such that the second safety syringe is positioned in alignment with the distal opening of the outer casing such that distal advancement of the second safety syringe will position the second safety syringe at least partially out of the distal opening on the distal surface of the outer casing.

In certain instances, the first safety syringe includes: the first outer casing including a first outer casing distal opening, a first inner surface, and a first track defined on the first inner surface. The first safety syringe also includes a first carrier, a first distal tube, a first biasing member, and a first ring. The first carrier is configured to support the first syringe in a position within the first outer casing. The first distal tube includes a first distal end, the first distal tube slidably engaged with the first carrier and configured to be in a first extended state and in a first retracted state, wherein, in the first retracted state, the first needle of the first syringe extends beyond the first distal end and the first outer casing distal opening. The first biasing member engaged with the first carrier and the first distal tube so as to bias the first distal tube in the first extended state. The first ring includes a first ring protrusion, the first ring engaged with the first distal tube and the first biasing member, the first ring protrusion positioned at least partially within the first track defined on the first inner surface.

In certain instances, the first safety syringe further includes a first plunger coupler biasing member that is engaged with the first plunger coupler and the first outer casing of the first safety syringe. Wherein the first plunger coupler biasing member is configured to release engagement with the first plunger couple upon overcoming a biasing force so as to permit distal advancement of the first plunger coupler relative to the first outer casing.

In certain instances, the biasing force associated with the first plunger coupler biasing member is greater than another biasing force associated with the first biasing member.

In certain instances, the at least one safety syringe includes the first safety syringe, a second safety syringe, a third safety syringe, and a fourth safety syringe.

In certain instances, the system further includes the first drug within the first barrel of the first syringe.

In certain instances, the cap includes a display device, the microcontroller in communication with the display device.

In certain instances, the first plunger support portion is a cap.

In certain instances, the dispensing device further includes an inner casing that is fitted at least partially within the inner volume of the outer casing, the inner casing being slidable relative to the outer casing.

In certain instances, distal advancement of the first safety syringe at least partially out of the distal opening of the outer casing causes distal advancement of the inner casing.

In certain instances, the first safety syringe further includes a ring that contacts the inner casing and causes the inner casing to advance distally when the first safety syringe is advanced distally.

Aspects of the present disclosure may include a method including providing the safety syringe system as described herein, and dispensing the first safety syringe at least partially from the dispensing device.

Aspects of the present disclosure may include a system including a safety syringe system. The safety syringe system includes a dispensing device and at least one safety syringe supported within the dispensing device and configured to be dispensed from the dispensing device. The dispensing device includes a carrier assembly supporting the at least one safety syringe and including at least one motor, a casing including a distal opening and enclosing the carrier assembly and the at least one safety syringe, and a computing device having a memory and one or more processors in communication with the at least one motor. The memory stores instructions that, as a result of being executed by the one or more processors, cause the system to perform the operations including actuating a first motor of the at least one motor to dispense a first safety syringe of the at least one safety syringe at least partially out of the distal opening of the casing.

In certain instances, actuating the first motor is permitted only at a predetermined time.

In certain instances, the predetermined time is when a dosage of a medicament within the first safety syringe is due for a user.

In certain instances, the dispensing device further includes a display screen in communication with the one or more processors.

In certain instances, the computing device is part of a microcontroller.

Aspects of the present disclosure can include a syringe safety system that includes a dispenser device and one or more safety syringes or injectors. Each of the safety syringes can include a casing configured to enclose a syringe having a barrel, a needle extending distally from the barrel, and a plunger insertable within the barrel to dispense a drug within the barrel.

Aspects of the present disclosure may include a syringe safety system including a dispensing device configured to enclose and dispense one or more syringes. The dispensing device includes an outer casing, a cap, a microcontroller, and a carrier assembly. The outer casing includes a sidewall defining an inner volume, a distal opening, and a proximal opening opposite the distal opening. The cap is configured to couple to and enclose the proximal opening of the outer casing. The microcontroller is configured to be housed within the outer casing and is in communication with at least one battery. The carrier assembly is configured to support the one or more syringes within the inner volume of the outer casing. The carrier assembly includes a drive system in communication with the microcontroller, the drive system is configured to provide movement of a first syringe of the one or more syringes from within the inner volume of the outer casing to at least partially out of the inner volume of the outer casing.

In certain instances, the drive system is configured to retract the first syringe back into the inner volume of the casing.

In certain instances, the first syringe includes a first barrel, a first needle extending distally from the first barrel, and a first plunger insertable within the first barrel to dispense a first drug within the first barrel, the first syringe being enclosed within a first outer casing.

In certain instances, the drive system includes a lead screw, a lead nut moveably positioned on the lead screw, and a motor rotatably coupled to the lead screw.

In certain instances, the lead nut operably couples with the first syringe supported by the carrier assembly, wherein actuation of the motor causes rotation of the lead screw and translation of the lead nut and the first syringe at least partially out of the inner volume of the outer casing.

In certain instances, the microcontroller restricts operation of the drive system until a pre-programmed release time. In certain instances, the cap includes a display screen in communication with the microcontroller, the display screen configured to display an indicator associated with the pre-programmed release time. In certain instances, when the pre-programmed release time occurs, the microcontroller permits operation of the drive system via a user interaction with at least one of the display screen or control mechanism on the dispensing device. In certain instances, the control mechanism is a button on the cap.

In certain instances, the one or more syringes include the first syringe, a second syringe, a third syringe, and a fourth syringe.

The casing of the safety syringe can include a casing distal opening, an inner surface, and a track defined on the first inner surface. Each of the safety syringe can further include a cartridge configured to support the syringe in a position within the casing.

Each of the safety syringe can further include a distal tube including a distal end, the distal tube slidably engaged with the cartridge and configured to be in a extended state and in a retracted state. When in the retracted state, the needle of the syringe extends beyond the distal end of the distal tube and the casing distal opening.

Each of the safety syringe can further include a biasing member, such as a torsional spring, engaged with the cartridge and the distal tube so as to bias the distal tube in the extended state.

Each of the safety syringe can further include a ring including a ring protrusion. The ring can be engaged with the distal tube and the biasing member. The ring protrusion can be positioned at least partially within the track defined on the inner surface of the casing. The track is designed to permit a single retraction of the distal tube and then a subsequent advancement, which is then locked in the extended state. This locking action including the structures of the track on the inner surface of the casing, among other features, is described in detail in PCT/US2022/023878, filed Apr. 7, 2022, which is hereby incorporated by reference in its entirety.

The dispensing device of the syringe safety system can include a casing, a cap, an electronics insert, and a cartridge assembly.

The casing can include a curvate sidewall defining an inner volume, a distal surface, a proximal opening opposite the distal surface, a distal opening extending through the distal surface, a casing motor coupled to the distal surface, and a gear rotatably coupled to the casing motor. The proximal opening may be threaded in order to rotatably receive the cap.

The cap can couple to and enclose the proximal opening of the casing. The cap can include a display device that shows the time remaining until the next dose of a drug from an injector is permitted. When the time for the dose of the next drug is due, the display device will indicate as such. The display device or a button on the cap can then be actuated by the user. The actuation of the button will advance the next safety syringe through the distal opening in the casing for use by the user.

The electronics insert can be received in the proximal opening of the casing. The electronics insert can include a microcontroller in communication with the display device, the button, and the two motors in the dispenser device.

The carrier assembly can be positioned within the inner volume of the casing. The carrier assembly can include a carrier clip or cylinder configured to support the at least one safety syringe around a central axis, a guide tube coupled to the cylinder and comprising a first track for guiding linear movement of the first injector, a lumen extending through the guide tube, a lead screw positioned within the lumen, a guide tube motor rotatably coupled to the lead screw (the guide tube motor secured to the guide tube and in electrical communication with the microcontroller), and a lead nut engaged with the lead screw and comprising a protrusion that is sized and shaped to be received in the first track to guide linear movement of the injector.

The carrier assembly can further include at least one set of battery connectors extending from the guide tube, which are configured to receive at least one battery (e.g., AA batteries, AAA batteries). The at least one battery is electrically connected to the microcontroller.

The carrier assembly can further include a plunger cap for each injector. The plunger cap can include a cylindrical body and a protrusion extending outwards from the cylindrical body. The protrusion can be received within the track of the guide tube. The protrusion defines a recess therein as well, which permits the protrusion of the lead nut to fit therein to guide linear (distal and proximal) movement of the plunger cap to depress the plunger of the syringe. The cylindrical body positioned to engage the plunger of the syringe. The protrusion of the lead nut is configured to fit within the recess on the plunger cap. That is, both the protrusion of the lead nut and the protrusion of the plunger cap are in the track of the guide tube such that linear movement of the lead nut via rotation of the lead screw by the guide tube motor is configured to cause distal advancement of the plunger cap and the injector at least partially out of the distal opening of the casing.

The motor within the casing engages with the distal end of the guide tube such that after an injector is used and proximally retracted back into the inner volume of the casing, the motor rotates the cartridge assembly so that the next injector is positioned for distal advancement out of the distal opening when such advancement is permitted by the microprocessor.

### BRIEF DESCRIPTION OF THE DRAWINGS

FIGS. 1A and 1B are isometric exploded views of a safety syringe system housing four safety syringes from a proximal end and a distal end, respectively.

FIGS. 1C and 1D are isometric views of the safety syringe system from a proximal end and a distal end, respectively.

FIGS. 2A and 2B are isometric views of an outer casing of the safety syringe system from a proximal end and a distal end, respectively.

FIGS. 3A and 3B are isometric views of an inner casing of the safety syringe system from a proximal end and a distal end, respectively.

FIGS. 4A and 4B are isometric views of a lid of the safety syringe system from a proximal end and a distal end, respectively.

FIGS. 5A and 5B are isometric views of a carrier assembly of the safety syringe system from a proximal end and a distal end, respectively.

FIGS. 5C and 5D are isometric views of a subset of parts of the carrier assembly from a proximal end and a distal end, respectively.

FIG. 5E is an isometric view of the lead screw and lead nut of the carrier assembly.

FIGS. 5F and 5G are isometric views of a carrier motor, and a lead screw motor, respectively.

FIGS. 6A and 6B are isometric views of a safety syringe of the safety syringe system from a proximal end and a distal end, respectively.

FIG. 6C is an isometric exploded view of the safety syringe containing a syringe therein.

FIGS. 6D and 6E are, respectively, isometric and side views of the safety syringe in a state of partial disassembly and prior to use.

FIGS. 6F and 6G are, respectively, isometric and side views of the safety syringe in a state of partial disassembly and with the distal tube retracted thereby exposing the needle.

FIGS. 6H and 6I are, respectively, isometric and side views of the safety syringe in a state of partial disassembly and with the distal tube retracted, the needle exposed, and the plunger depressed.

FIGS. 6J and 6K are, respectively, isometric and side views of the safety syringe in a state of partial disassembly and with the plunger depressed and the distal tube re-extended into a locking position.

FIGS. 6L and 6M are, respectively, isometric and side views of the distal tube and the lock ring.

FIGS. 7A through 7D are isometric views of the carrier assembly with a single safety syringe supported thereon in various states of operation.

FIGS. 8A and 8B are a side view and a side cross-sectional view of the safety syringe system enclosing multiple safety syringes in a first state of operation.

FIGS. 8C and 8D are a side view and a side cross-sectional view of the safety syringe system enclosing multiple safety syringes in a second state of operation.

FIGS. 8E and 8F are a side view and a side cross-sectional view of the safety syringe system enclosing multiple safety syringes in a third state of operation.

FIGS. 8G and 8H are a side view and a side cross-sectional view of the safety syringe system enclosing multiple safety syringes in a fourth state of operation.

FIG. 9 illustrates an example of a suitable computing and networking environment.

### DETAILED DESCRIPTION

Aspects of the present disclosure can include a safety syringe system (“SSS”) including a dispenser device and safety syringes (also referred to as injectors or pre-filled safety syringes) housed within the dispenser device. Methods of use are also disclosed. More particularly, the disclosure relates to SSS with time-controlled release of the injectors that can be manually actuated to deliver a dose of a drug. In one embodiment, the safety syringes described herein can be the same or similar to the safety syringes in PCT/US 2022/023878, filed Apr. 7, 2022, which is incorporated by reference herein in its entirety for all purposes. The SSS provides for an innovative way to allow for time-gated access of multiple injections. In certain embodiments, the SSS is a dispenser housing four safety syringes; however, the SSS can house and dispense any number of safety syringes from one to ten. The SSS utilizes an outer shell or casing with built in electronic components, which will accept a cartridge carrier holding multiple injectors or cartridges. The SSS offers a time-gated sequential locking mechanism, which allows individual injectors to be utilized only at pre-set dose times. This prevents injection medication mis-dosing (i.e., over-dosing, under-dosing) by ensuring the user is only able to administer the injection designated for that pre-set time interval. SSS allows for the implementation of a single type of medication or multiple different medication types as it can accept various injectors allowing for specific medication regimens requiring intermittently dosed multiple medications.

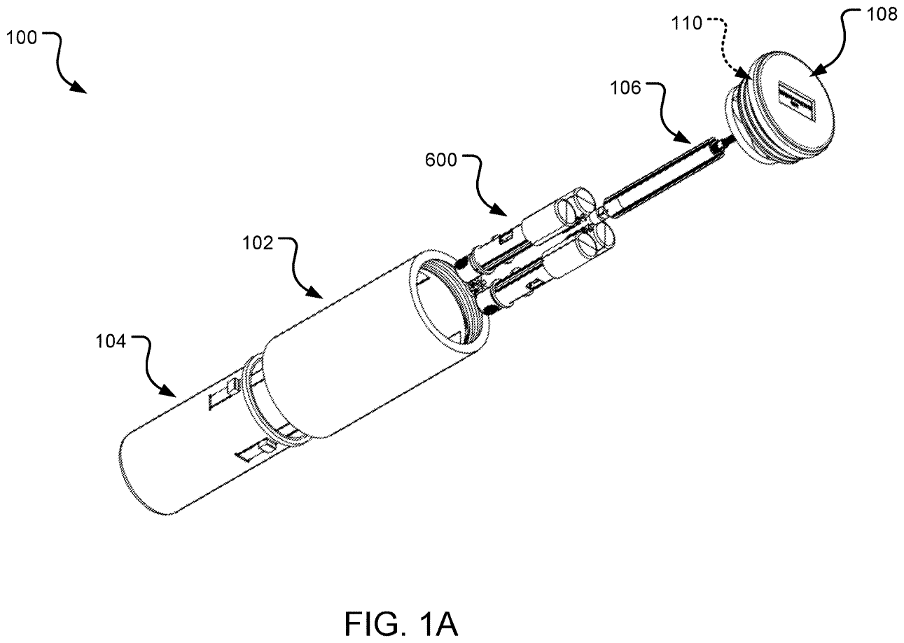

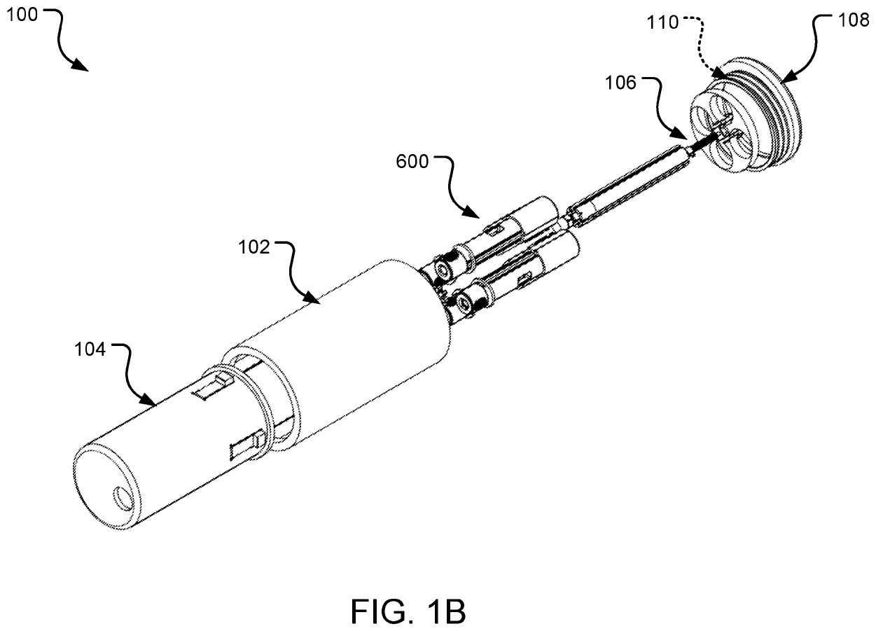

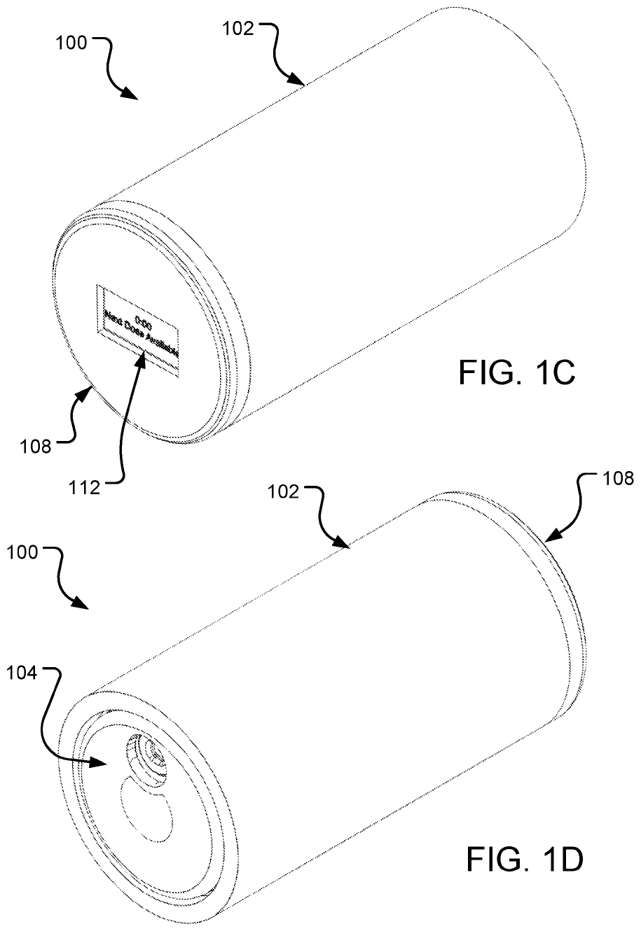

FIGS. 1A and 1B are isometric exploded views of an SSS 100 housing four safety syringes 600 from a proximal end and a distal end, respectively. As seen in the figures, the SSS 100 includes an outer casing 102 an inner casing 104 that is slidable relative to the outer casing 102, a carrier assembly 106 for supporting the safety syringes 600 within the outer casing 102 and inner casing 104, a cap 108 that can be threadably secured to the proximal end of the outer casing 102, and a microcontroller 110 positioned on the underside of the cap 108. The components of the SSS 100 that facilitate dispensing of the safety syringes 600 may be described as a dispensing device herein. That is, as described herein, the outer casing 102, the inner casing 104, the carrier assembly 106, the cap 108 and the microcontroller 110 may be collectively referred to as a dispensing device.

Medical terms of direction such as distal and proximal will be used herein to aid in the understanding of the technology and the methods described herein. With respect to the SSS 100 in FIGS. 1A and 1B, among others, the proximal end generally refers to the side of the device in the direction of the cap 108, whereas the distal end generally refers to the opposite direction and towards the end of the inner casing 104 where the tips of needle of the safety syringes 600 extend.

FIGS. 1C and 1D are isometric views of the SSS 100 from a proximal end and a distal end, respectively. FIG. 1C shows the cap 108 threadably secured to the outer casing 102. As seen in the figure, the cap 108 includes display screen 112 that reads “Next Dose Available” and a timer that indicates “0:00”. This would indicate that the next dose of medication from the safety syringe is available now. The timer may be configured to count down a programmed time such that the device will not dispense a safety syringe until the timer reaches 0:00. When the timer reaches 0:00, the user may press a button on the display screen 112 or on the cap (not seen) to dispense the safety syringe that is due at the time. FIG. 1D shows the inner casing 104 slidably positioned within the outer casing 102. In certain embodiments, the combination of the inner casing 104 and the outer casing 102 may be replaced by a single casing such as shown and described in U.S. Provisional Ser. No. 63/378,664, filed Oct. 6, 2022, which is hereby incorporated by reference herein in its entirety for all purposes.

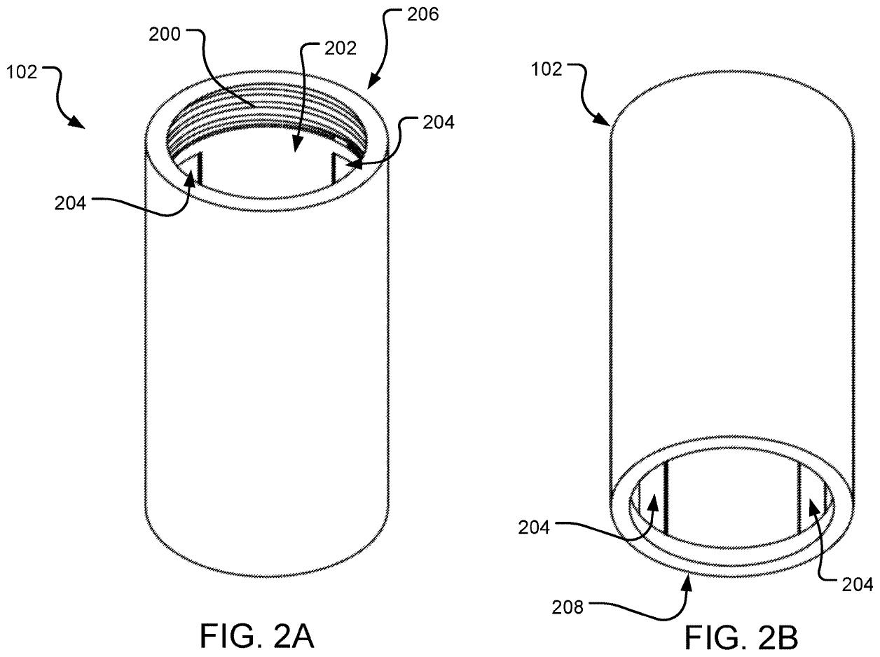

FIGS. 2A and 2B are isometric views of the outer casing 102 of the SSS 100 from a proximal end and a distal end, respectively. As seen in FIG. 2A, the outer casing 102 is a tube that includes threads 200 on an inner surface 202 of the proximal end. The inner surface 202 also includes tracks 204 extending longitudinally down the tube. The tracks 204 guide the longitudinal movement of the inner casing 104. The outer casing 102 includes a proximal opening 206 and a distal opening 208.

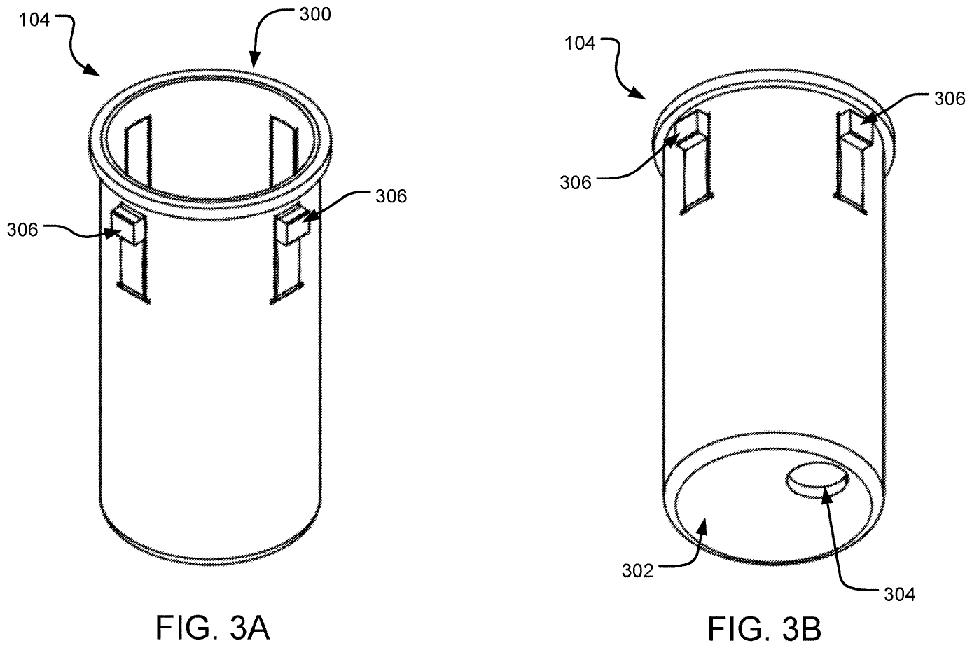

FIGS. 3A and 3B are isometric views of the inner casing 104 of the SSS 100 from a proximal end and a distal end, respectively. As seen in the figures, the inner casing 104 includes a proximal opening 300 and a distal surface 302 with an off-center distal opening 304 for the safety syringes (not shown) to extend there through. On a side of the inner casing 104 are depressible tabs 306 that can move inward. These tabs 306 fit within the tracks on the inner surface of the outer casing such that the inner casing 104 can translate within the outer casing 102.

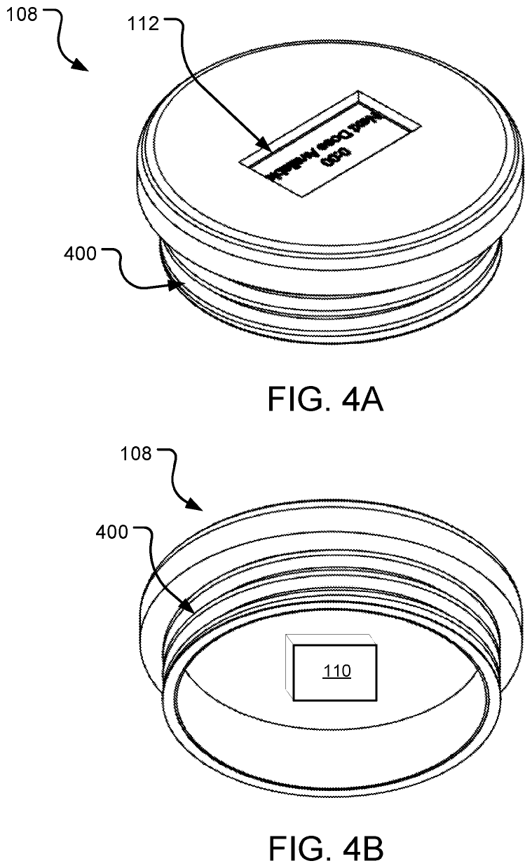

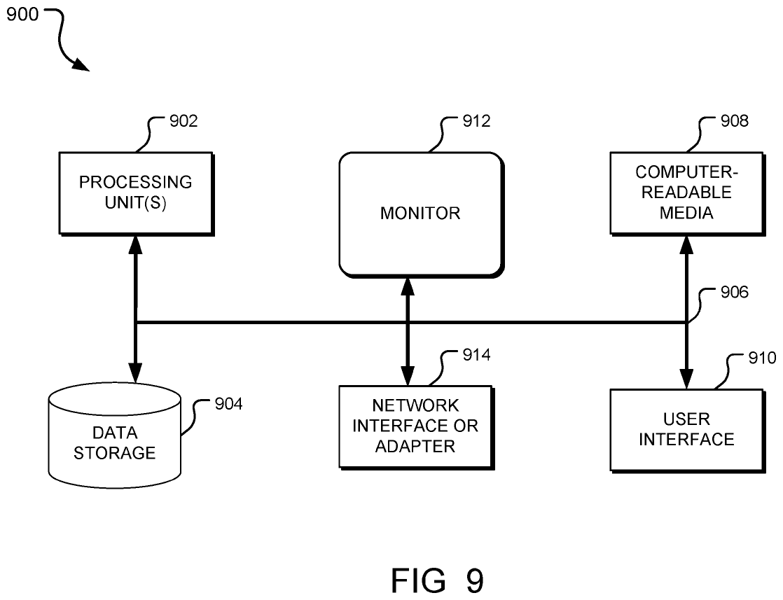

FIGS. 4A and 4B are isometric views of the cap or lid 108 of the SSS 100 from a proximal end and a distal end, respectively. As seen in FIG. 4A, the cap 108 includes threads 400 for coupling with the threads on the outer casing. The cap 108 also includes the display screen 112 for displaying a timer, which shows “0:00” in the figure, and an indicator showing whether or not it is time to dispense a safety syringe or not. In the figure, the display screen 112 says “Next Dose Available 0:00” indicating that it is time to dispense a safety syringe. As seen in FIG. 4B, the microcontroller 110 is coupled to the underside of the cap 108. As will be described with reference to FIG. 9, the microcontroller 110 can operate the timer and can operate to permit a user to dispense one of the safety syringes (i.e., via a button or through the display screen) when the timer reaches a certain time (e.g., 0:00).

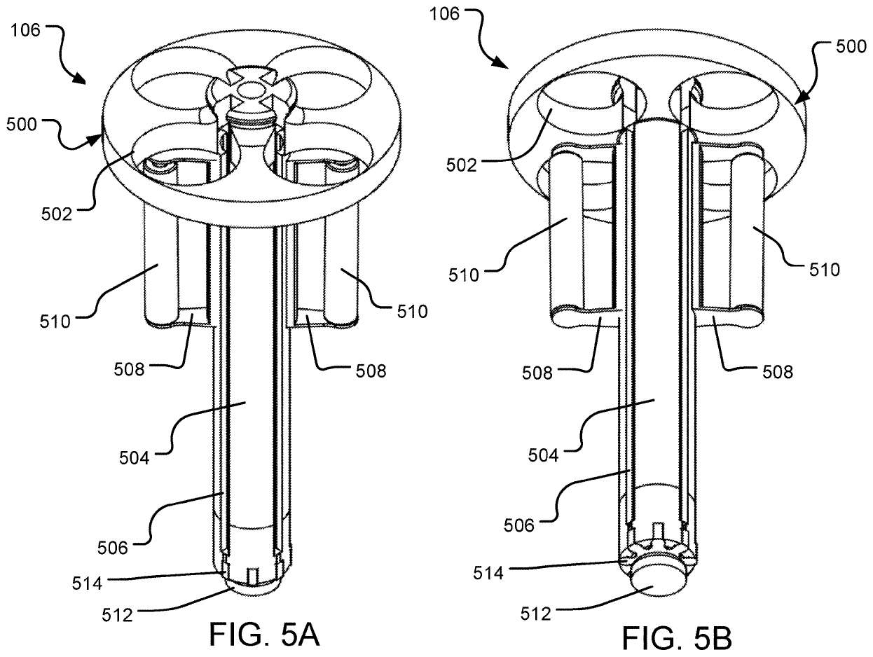

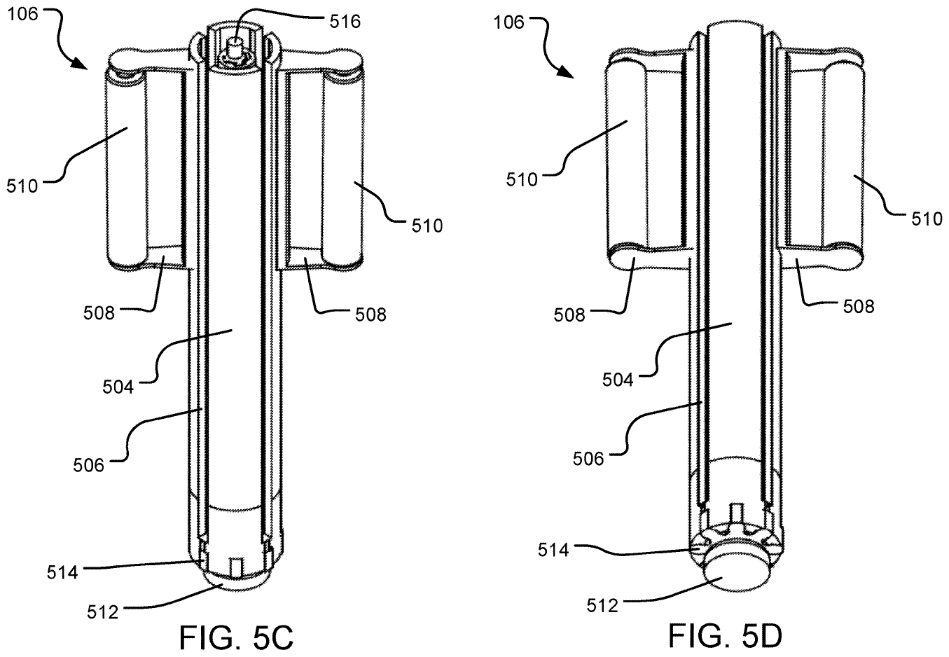

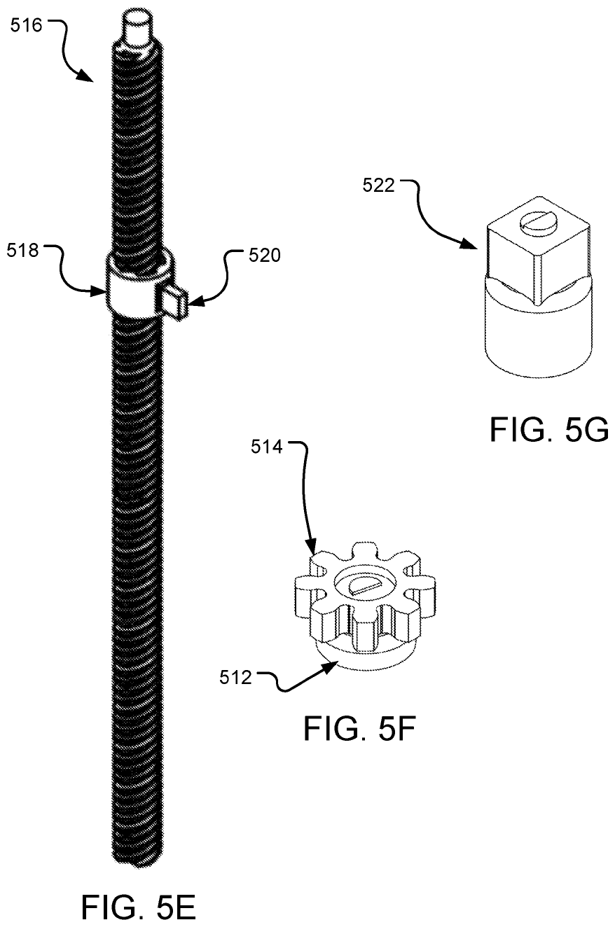

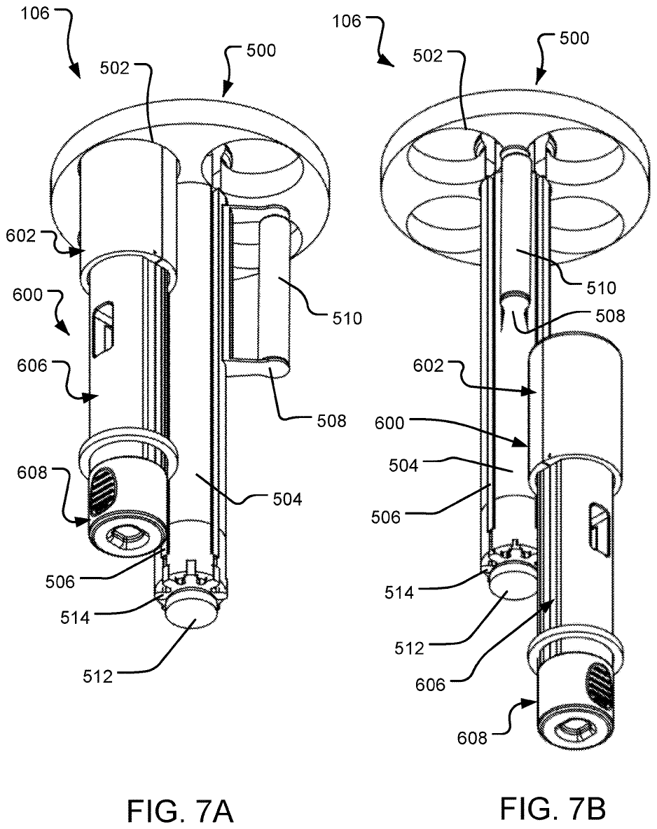

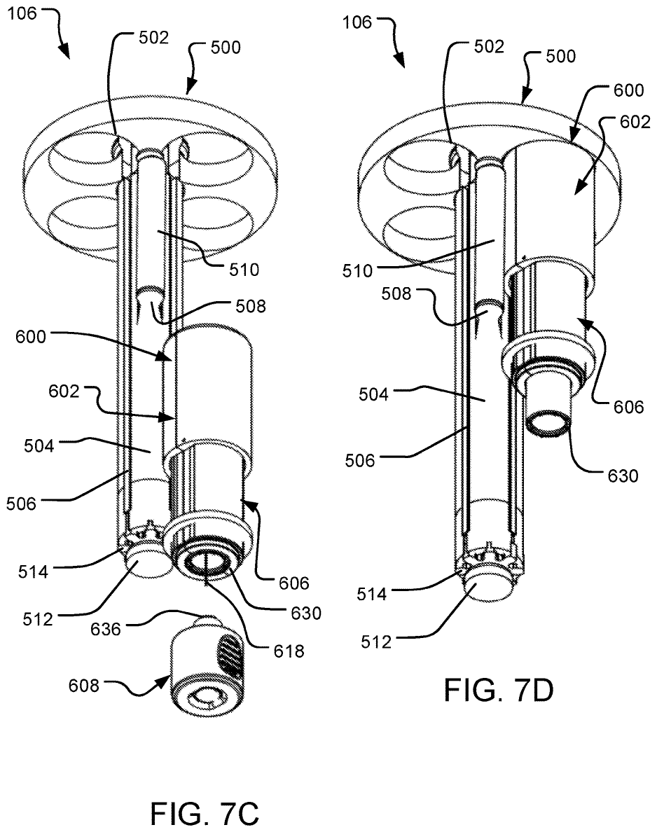

FIGS. 5A and 5B are isometric views of the carrier assembly 106 of the SSS 100 from a proximal end and a distal end, respectively. The carrier assembly 106 includes multiple components for supporting up to four safety syringes 600 and dispensing the syringes 600 to a user. The carrier assembly 106 with the supported safety syringes 600 are shown in detail in FIGS. 7A-7D. Referring to FIGS. 5A and 5B, the carrier assembly 106 includes a carrier clip or cylinder 500 having four chambers 502 for supporting and aligning a safety syringe 600. Each of the chambers 502 are radially aligned equidistant from each other. A guide tube 504 is coupled in a center slot of the cylinder 500 and extends longitudinally. The guide tube 504 includes four slots or tracks 506 that extend linearly the length of the tube 504. A pair of batter connectors 508 are connected to the guide tube 504 in a position that is between pairs of chambers 502. The battery connectors 508 may be electrically coupled/in communication with the microcontroller. In the illustrated embodiment, the batteries 510 connected to the battery connectors may be commercially available AA or AAA batteries.

The carrier assembly 106 also includes a carrier motor 512 and a gear 514 coupled with the motor 512, which are shown up close in FIG. 5F. The carrier motor 512 may be electrically coupled/in communication with the microcontroller and the batteries 510. The gear 514 connected to the carrier motor 512 fits within an end of the guide tube 504 and is configured to rotate the tube 504 when actuated by the microcontroller. Rotation of the guide tube 504 is done to align one of the safety syringes with the opening on the distal surface of the inner casing so that it can be dispensed to the user.

FIGS. 5C and 5D are isometric views of a subset of parts of the carrier assembly 106 from a proximal end and a distal end, respectively. As seen in FIG. 5C, in particular, a lead screw 516 is positioned within the guide tube 504.

The lead screw 516 is shown in FIG. 5E. A lead nut 518 having a protrusion 520 is threadably coupled to the lead screw 516. Although not seen in FIGS. 5C and 5D, a lead screw motor 522, shown in FIG. 5G, is coupled to the distal end of the lead screw 516 and is also housed within the guide tube 504. The lead screw motor 522 is designed to rotate the lead screw 516, and the protrusion of the lead screw nut 518 is sized and shaped to fit within the tracks of the guide tube. As such, when the lead screw 516 is rotated, the lead nut 518 is translated distal-proximal along the lead screw via guidance by the track of the guide tube. As will be described subsequently, translation of the lead nut 518 causes the safety syringe 600 to be dispensed out of the distal opening of the inner casing for use by the user. The carrier motor 512, the gear 514, the guide tube 504, the lead screw 516, the lead nut 518, and the lead screw motor 522 are all part of the drive system for the SSS 100. The drive system effectuates the movement of the safety syringes 600 from within the inner volume of the dispensing device to out the distal end of the device.

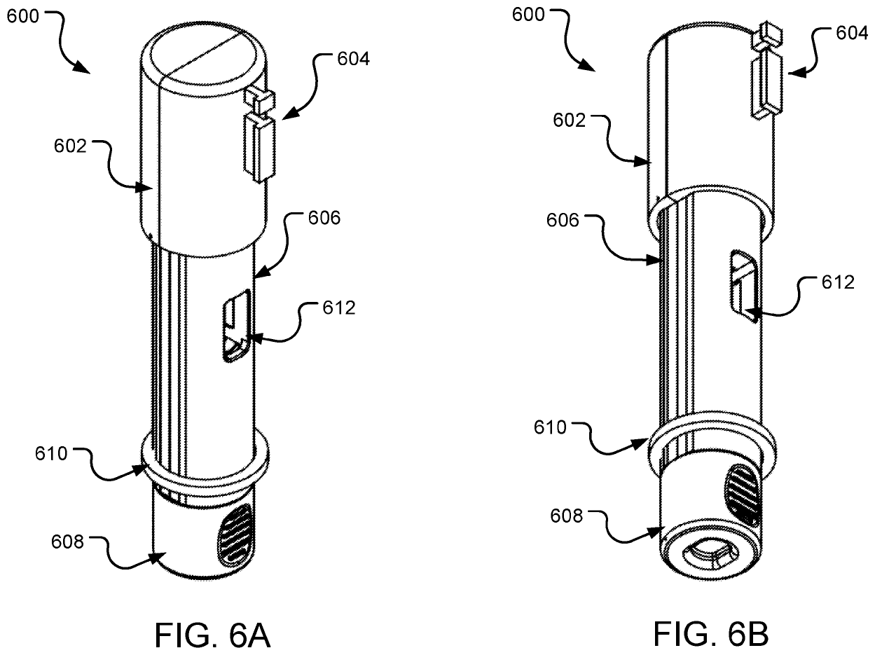

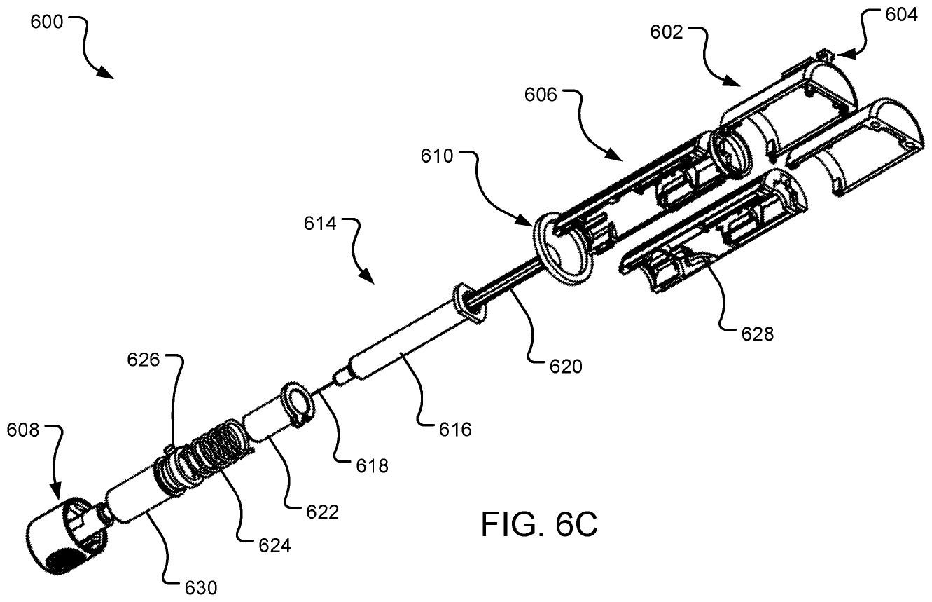

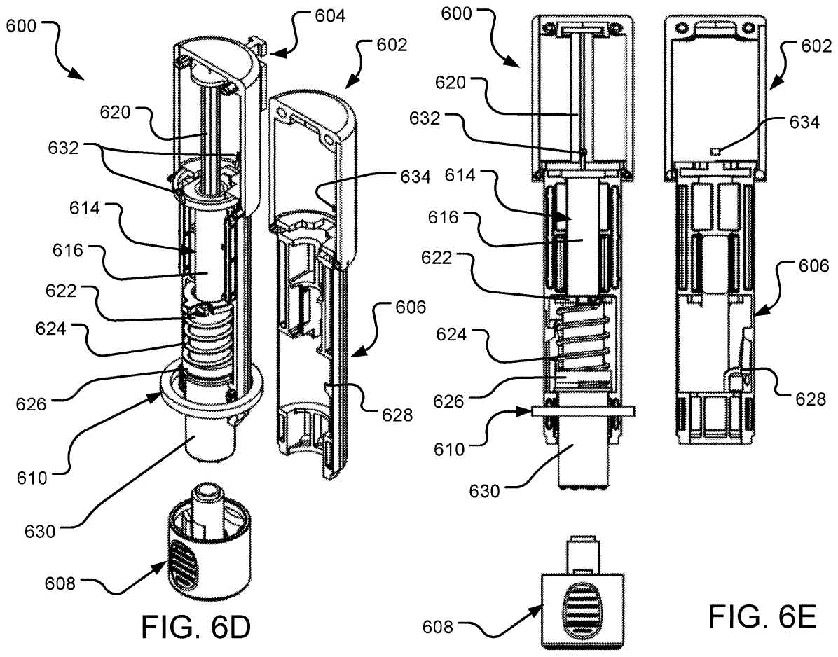

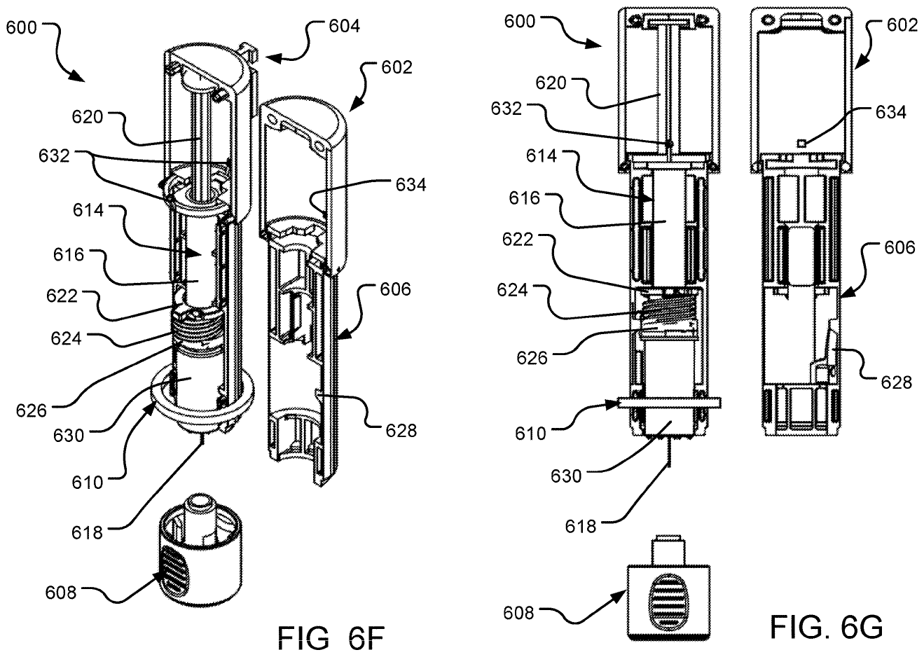

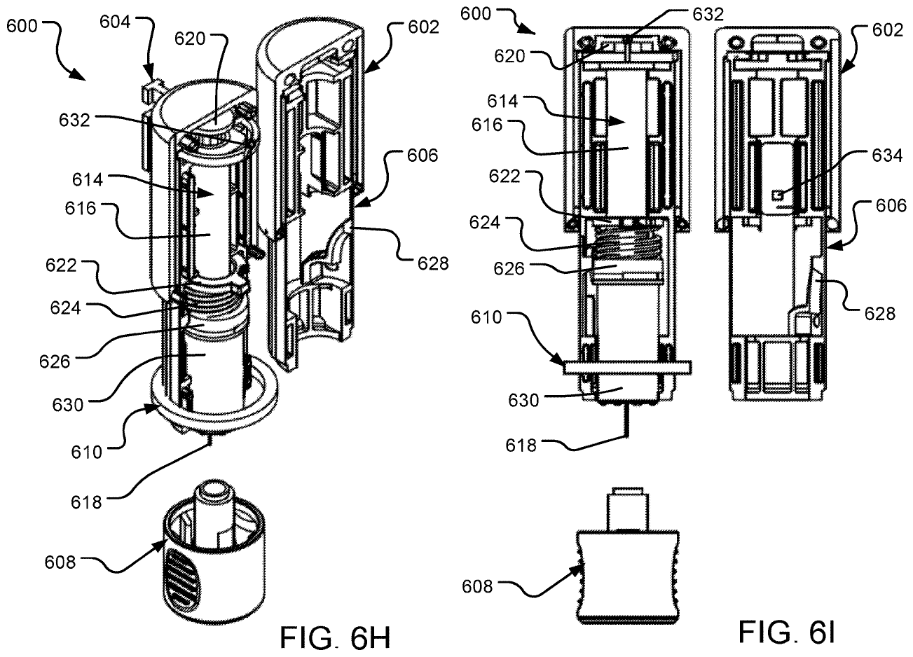

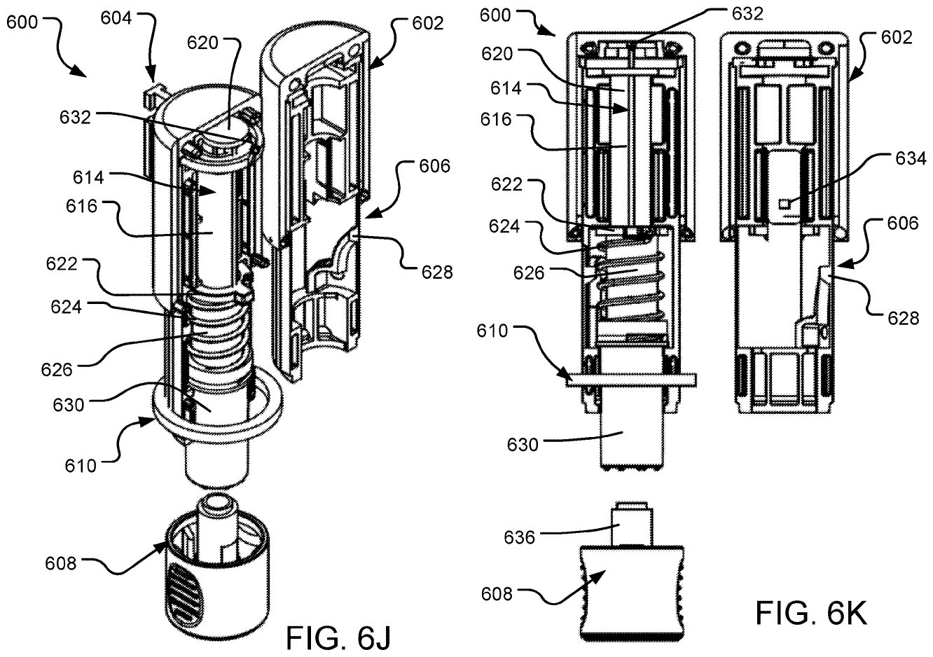

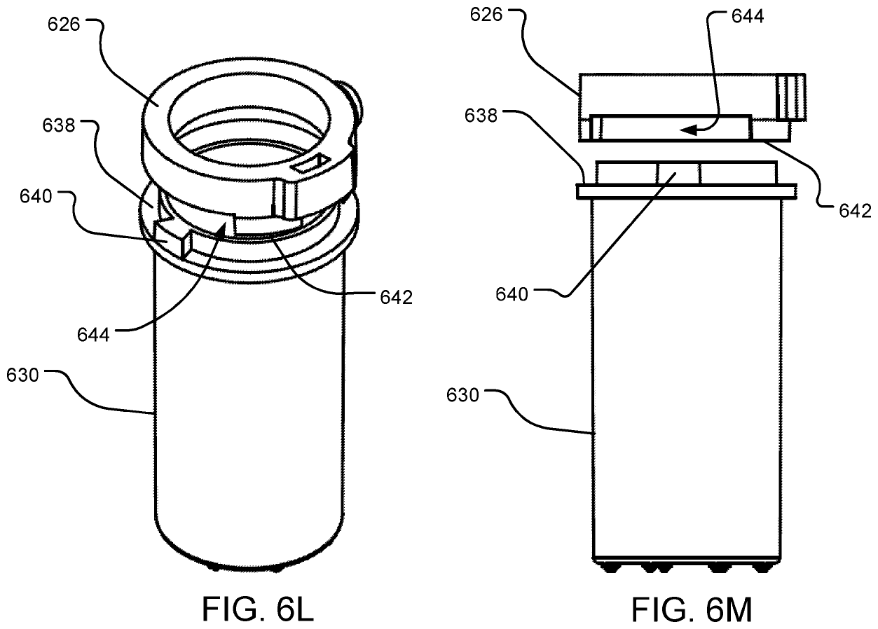

FIGS. 6A and 6B are isometric views of the safety syringe 600 of the safety syringe system 100 from a proximal end and a distal end, respectively. As seen in the figures, the safety syringe 600 includes a plunger cap 602 having T-shaped guides 604 extending from a side of the cap 602. The guides 604 are sized and shaped to fit within the track of the guide tube. While the plunger cap 602 is shown as a cylindrical structure that fits over the plunger of the syringe, alternative designs are contemplated herein. For instance, other structures that attach to the plunger of the syringe without otherwise covering the proximal end of the plunger are possible. The safety syringe 600 further includes a casing 606, which is a two-part shell that can be coupled together for ease of manufacturing. The safety syringe 600 further includes a needle cap 608 at a distal end thereof. The safety syringe 600 further includes a ring 610 that fits around the casing 606 and is designed to push the inner casing distally relative to the outer casing when the safety syringe 600 is distally advanced via rotation of the lead screw, which causes longitudinal translation of the lead nut, which pushes against the guides 604 of the plunger cap 602. As seen in FIGS. 6A and 6B, the casing includes a window 612 for viewing the contents of the syringe (not seen), which contains a medicament therein.

FIG. 6C is an isometric exploded view of the safety syringe 600 containing a syringe 614 therein. The syringe 614 includes a barrel 616, a needle 618, and a plunger 620. The safety syringe 600 further includes a cartridge 622 that supports the barrel 616 of the syringe 614. The cartridge 622 has a flange at its proximal end that fits within a corresponding recess on the casing 606 in order to hold it in position. The safety syringe 600 further includes a biasing member 624, which is a spring in this embodiment. The cartridge 622 is positioned within the coil of the biasing member 624 and attached to the flange of the cartridge 622. An opposite end of the biasing member 624 is coupled to a lock ring 626 that has a protrusion that fits within a track 628 on the inner surface of the casing 106 and that guides retraction and extension of the distal tube 630. The interaction of the lock ring 626 and the track 628 can be the same or similar to that described in FIGS. 13A-13D, among other places, in PCT/US2022/023878, filed Apr. 7, 2022, which is incorporated by reference herein in its entirety for all purposes. The track 628 includes a recessed surface in the casing 106 in which the protrusion of the lock ring 626 fits within. The track 628 permits a single retraction of the distal tube 630, which exposes the needle, and then an extension of the distal tube 630 back over the needle. Once the distal tube 630 is extended back over the needle, the protrusion of the lock ring 626 is positioned at a point in the track 628 that restricts subsequent proximal retraction of the distal tube 630. The biasing member 624 provides both a biasing force that opposes the proximal retraction of the distal tube 630 and also a torsional force that guides the lock ring 626 along the track 628.

The distal tube 630 is a tube with proximal and distal openings. The distal tube 630 engages with the lock ring 626 and a certain amount of rotation is permitted between the two components. The distal tube 630 is capable of retracting relative to the cartridge 622 by overcoming the biasing force of the biasing member 624 (compressing the spring) such that the cartridge 622 is received and advanced within the proximal opening of the distal tube 630. Given that the needle 618 and barrel 616 of the syringe 614 and the cartridge 622 are stationary relative to the casing 106, the needle 618 is exposed out of the distal opening of the distal tube 630 when the distal tube 630 is retracted.

FIGS. 6D and 6E are, respectively, isometric and side views of the safety syringe 600 in a state of partial disassembly and prior to use. That is, the cap 602 and the casing 604 are two-piece constructions that are opened for viewing purposes. This is the orientation of the safety syringes 600 as they would be housed within the SSS 100. That is, the syringes 614 are loaded with a drug and the plungers are extended and ready for depression. The distal tube 630 is extended over the needle (not shown) and ready to be retracted upon a force applied against the distal end of the distal tube.

FIGS. 6F and 6G are, respectively, isometric and side views of the safety syringe 600 in a state of partial disassembly and with the distal tube 630 retracted thereby exposing the needle 618. The distal tube 630 is initially biased in an extended state (FIGS. 6D and 6E). Upon applying a proximal force on the distal tube 630 via pressing the tube 630 against an injection site (e.g., user's arm or leg) causes the distal tube 630 compress the biasing member 624 and retract the distal tube 630. As the distal tube 630 retracts the needle 618 is exposed, as seen in FIGS. 6F and 6G. Thus, when in use, the needle 618 is inserted into the injection site as the distal tube 630 retracts. This is a manual operation where the user controls the speed of injecting the needle into the injection site and the subsequent delivery of the medication.

FIGS. 6H and 6I are, respectively, isometric and side views of the safety syringe 600 in a state of partial disassembly and with the distal tube 630 retracted, the needle 618 exposed, and the plunger 620 depressed. Once the distal tube 630 is retracted and the needle 618 is exposed (and in the injection site of the patient), the medication can be dispensed from the syringe 614. To dispense the medication, the plunger 620 and the attached plunger cap 602 are depressed relative to the barrel 616 of the syringe 614. Since the safety syringe 600 is partially retained within the inner and outer casing 104, 102 of the SSS 100, the user does not have access to the plunger cap 602 to depress it (i.e., the plunger cap 602 is held in position via the lead screw nut of the carrier assembly). Instead, the user applies a continuous proximal force on the distal tube 630 to initially cause the distal tube 630 to retract and expose/inject the needle 618 into the injection site, and then to cause the casing 606, along with the enclosed cartridge 622, distal tube 630, biasing member 624, needle 618 and barrel 616 of the syringe 614, among the other components, to proximally retract into the cavity of the plunger cap 602, which causes the plunger 620 to push the medication through the barrel 616 of the syringe 614 and dispense the medication.

To effectuate the distal tube 630 being able to be retracted into the casing 606 without the casing 606 retracting into the plunger cap 602, a frictional engagement between the casing 606 and the plunger cap 602 has a biasing force that is greater than that of the biasing member 624. That is, a smaller force is needed to proximally retract the distal tube 630 into the casing 606 and a relatively larger force is needed to retract the entire casing 606 into the plunger cap 602. This results in the distal tube 630 retracting first (and injection of the needle) and injection of the medication happening second. In the illustrated embodiment, the frictional engagement between the casing 606 and the plunger cap 602 is a tab 632 on the casing 606 that is initially fitted within a divot or indent 634 formed in the plunger cap 602, as seen in FIGS. 6F and 6G. The tab 632 is biased in an outward direction (i.e., towards the indent 634 in the plunger cap 602). When the casing 606 is forced proximally relative to the plunger cap 602, the tab 632 moves inward and out of the indent 634. The plunger cap 602 can then converge with the casing 606.

It is contemplated herein that other mechanisms could be utilized to facilitate the casing 606 retracting into the plunger cap 602. For instance, U.S. Provisional Ser. No. 63/378,664 , filed Oct. 6, 2022, which is hereby incorporated by reference herein in its entirety for all purposes, describes a latch mechanism that releases the plunger cap 602 and permits movement between the plunger cap 602 and the casing 606. Such a mechanism can be employed in the embodiments shown and described herein without limitation.

FIGS. 6J and 6K are, respectively, isometric and side views of the safety syringe 600 in a state of partial disassembly and with the plunger 620 of the syringe 614 depressed and the distal tube 630 re-extended into a locking position. After the medication is injected (FIGS. 6H and 6I), the user can pull the safety syringe 600 away from the injection site. This causes the distal tube 630 to re-extend into a locked position with the distal tube 630 shielding the needle. At this point, the safety syringe cap 608 (which is shown with the needle cap 636 attached thereto) can be reattached to the distal tube 630 and needle. At this point, the safety syringe 600 can be retracted into the SSS 100, as will be discussed subsequently.

FIGS. 6L and 6M are, respectively, isometric and side views of the distal tube 630 and the lock ring 626. The lock ring 626 is positioned on the top of the distal tube 630 and the two components are permitted a limited amount of rotation between them. The top surface of the distal tube 630 includes cylindrical rim 638 with a stop feature 640 on the rim 638. The lock ring 626 includes a bottom surface 642 with a recessed area 644 that is sized and shaped to receive the stop feature 640 when the lock ring 626 is placed on the top surface of the distal tube 630. With the stop feature 640 of the distal tube 630 within the recessed area 644 of the lock ring 626, the two components are permitted a limited amount of rotation relative to each other. In this illustrated embodiment, the rotation is limited to about a quarter turn. In other embodiments the rotation of the two components is limited to about a half turn. In other embodiments the rotation of the two components is limited to about three quarters of a turn. In other embodiments the rotation of the two components is not limited; in such an embodiment, the distal tube 630 is devoid of the stop feature 640.

FIGS. 7A through 7D are isometric views of the carrier assembly 106 with a single safety syringe 600 supported thereon in various states of operation. The outer casing 102, inner casing 104, and cap 108 are not shown in these figures for ease of viewing purposes. FIG. 7A depicts the safety syringe 600 supported in the carrier assembly 106 in an initial state with the plunger cap 602 of the safety syringe 600 positioned within the chamber 502 of the cylinder 500. In this state, the safety syringe 600 encloses a syringe (not visible in FIGS. 7A and 7B) that is loaded with a medication. The protrusion 604 on the plunger cap 602 is positioned within the guide track 506 of the guide tube 504 such that it can be moved longitudinally along the track 506 from the proximal position, shown in FIG. 7A, to a distal position, shown in FIG. 7B.

To move from the proximal position to the distal position, the guide tube motor 512 is rotated (if necessary) such that the appropriate safety syringe 600 is oriented coaxial with the distal opening on the distal surface of the inner casing so that it can be dispensed out of the distal opening. Once rotated to the correct orientation, the lead nut (not seen) is positioned such that the protrusion of the lead nut is positioned within the guide track 506 for appropriate safety syringe 600. The lead screw can be rotated in a direction such that the lead nut, which is positioned between the protrusions 604 on the plunger cap 602, translates distally. Distal movement of the lead nut causes the safety syringe 600 to move distally to the position shown in FIG. 7B.

In the distal position, as shown in FIG. 7B, the needle cap 608 and syringe cap 636 (coupled together) can be removed from the distal tube 630 and the syringe 614. Then, as shown in FIG. 7C, the distal tube 630 can be applied to the injection site. Pushing the distal tube 630 onto the injection site causes the distal tube 630 to retract and the needle to be exposed (in the injection site), and then the casing 606 to be retracted into the cavity of the plunger cap 602 in order to dispense the medication into the injection site.

After the medication is injected, the user can press a button on the SSS 100 (i.e., on the display screen or a button on the cap) and the safety syringe 600 can be retracted back into the casings of the SSS 100. Retraction of the safety syringe 600 is shown in FIG. 7D. As seen in the figure, the distal tube 630 has been re-extended into a locking position. Retraction of the safety syringe 600 is accomplished by rotation of the lead screw (in the opposite direction), which causes the lead nut to move proximally within the guide track 506 of the guide tube 504 until the safety syringe 600 is back in the proximal position.

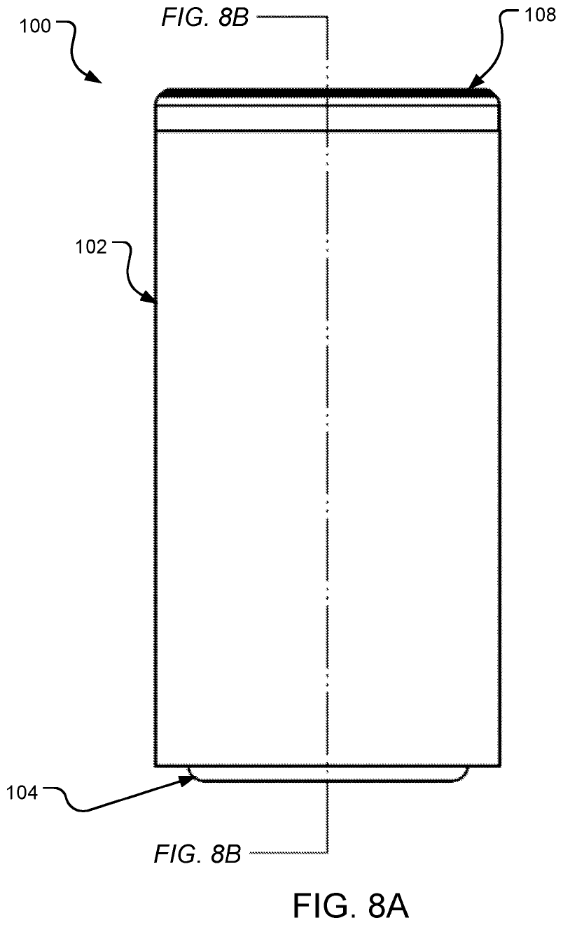

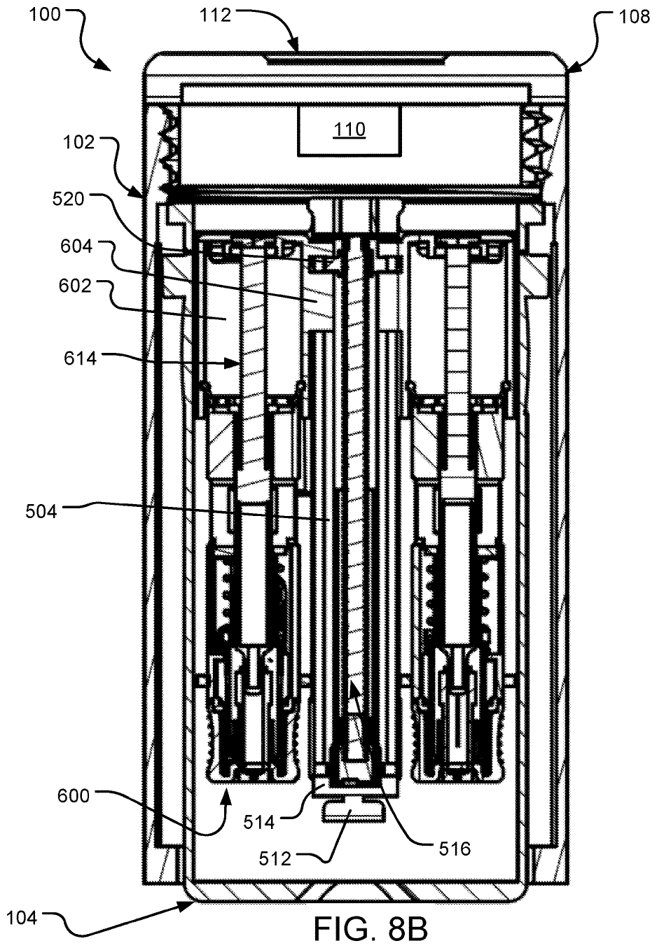

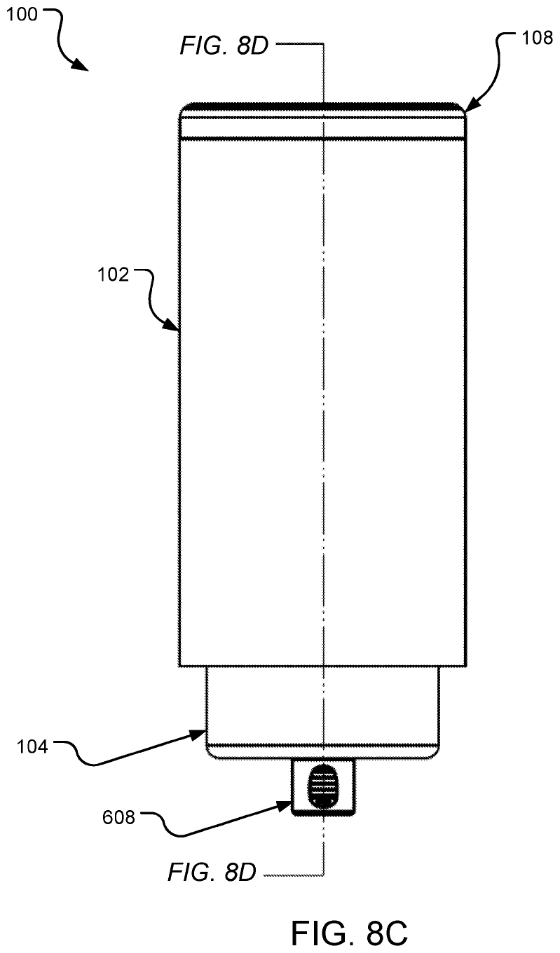

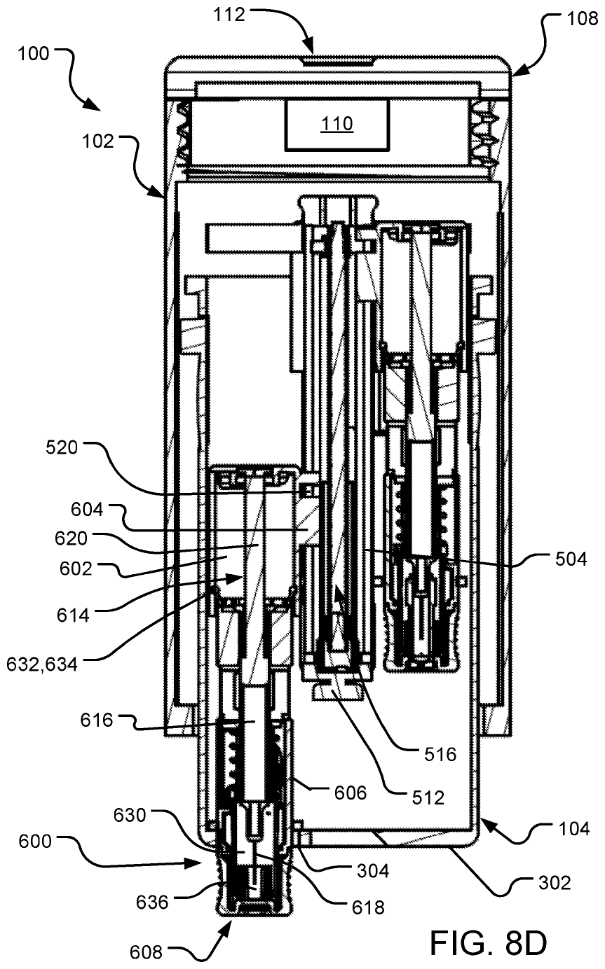

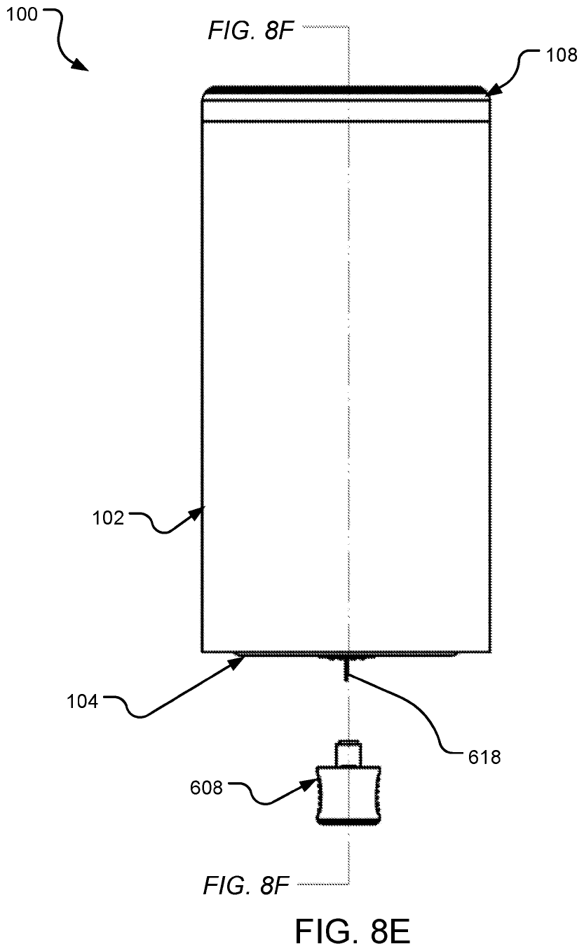

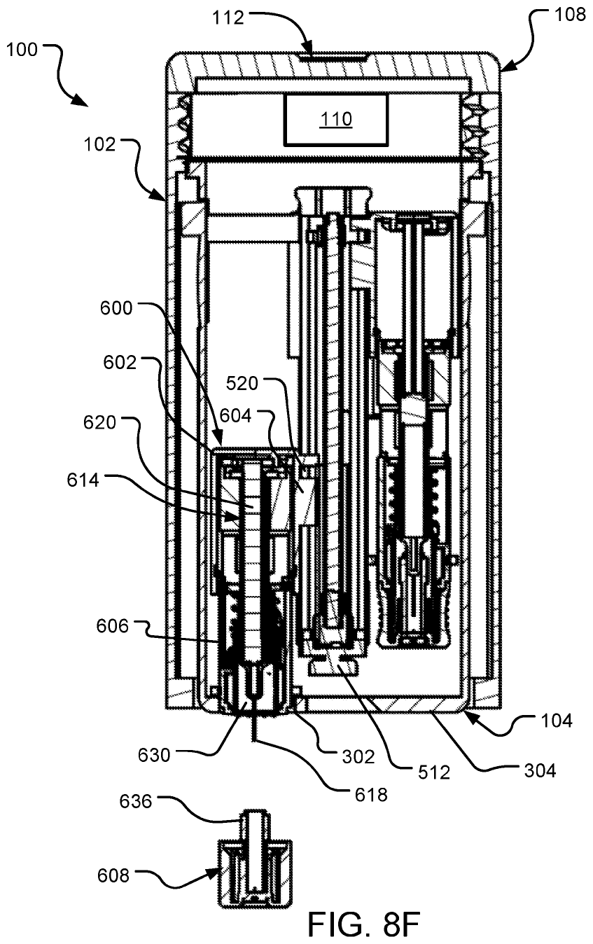

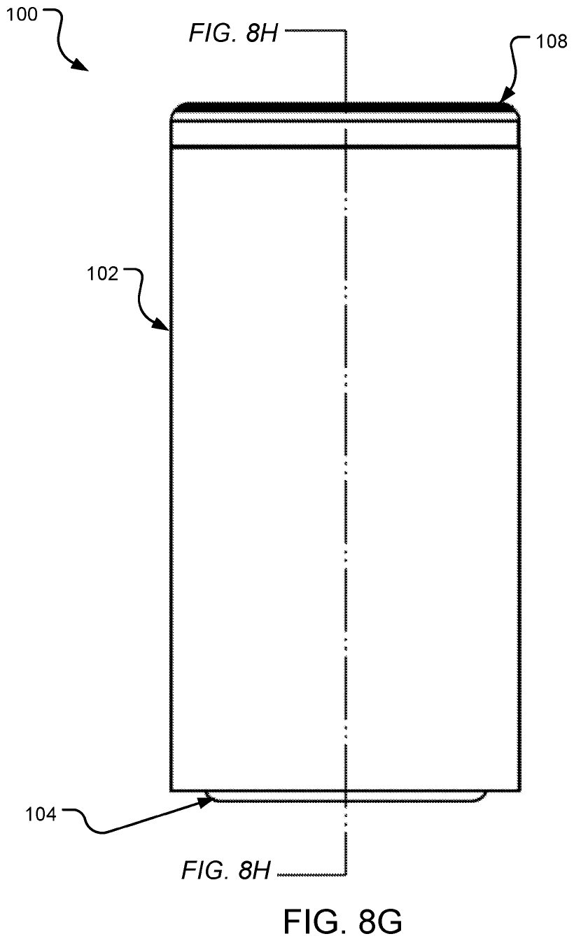

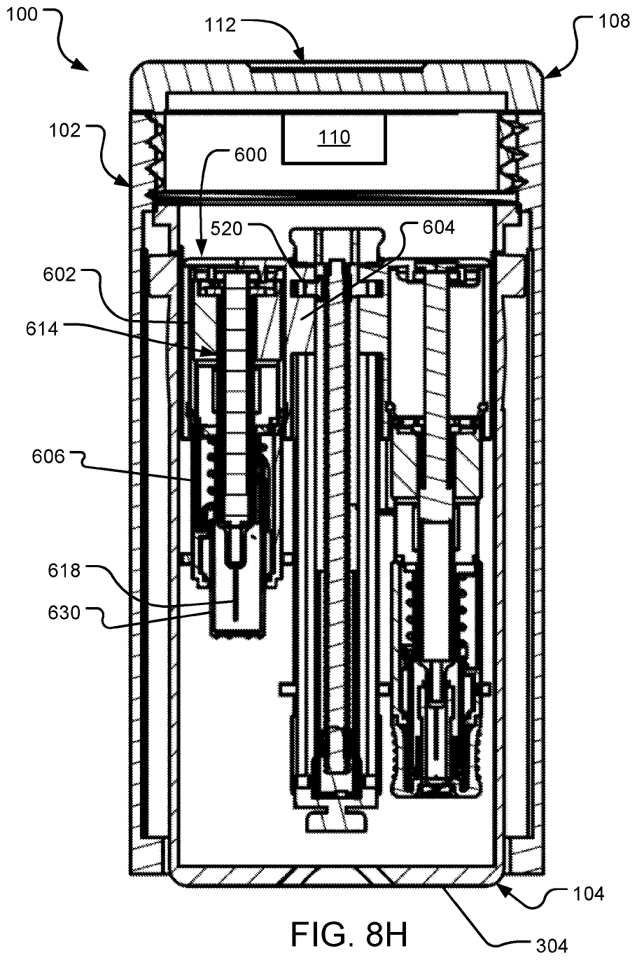

FIGS. 8A and 8B are a side view and a side cross-sectional view of the safety syringe system 100 enclosing multiple safety syringes 600 in a first state of operation. This is the state of the SSS 100 that may be provided to a user of the medication. Once the display screen indicates that a medication is due, the display screen or a button on the cap 108 may be actuated to dispense the medication that is due. If the medication that is due is in a safety syringe 600 that is not aligned with the distal opening on the inner sleeve 104, then the guide tube motor 512 may be rotated till the appropriate safety syringe 100 is positioned over the distal opening. Then, the lead nut will be positioned in the guide track 506 in a position between the protrusions 604 on the plunger cap 602. Then, the lead screw motor will be actuated to turn the lead screw resulting in linear movement of the lead nut and coupled plunger cap 602 in a distal direction. Movement of the plunger cap 602 causes movement of the entire safety syringe 600 such that the distal end of the safety syringe 600 is exposed out of the distal opening of the inner casing 104.

FIGS. 8C and 8D depict a second state of operation of the safety syringe system 100 where one of the safety syringes 600 has been translated distally via actuation of the lead screw motor 512 such that the distal tube 630 is extended out of the distal opening 304 of the distal surface 302 of the inner casing 104. As can be seen in the figures, the inner casing 104 has been pushed distally by the ring 610 around the casing 606 of the safety syringe 600. At this point, the user can remove the cap 608 and syringe cap 636. Then, the user can apply the distal tube 630 against the injection site (e.g., leg, arm), which will cause proximal retraction of the distal tube 630 and insertion of the needle 618 into the injection site. Continued pressure on the distal tube 630 and, more generally, the distal surface 302 of the inner casing 104, pushes the inner casing 104 and the casing 606 of the safety syringe 600 upwards (proximally) and into the plunger cap 602, which forces the plunger 620 to dispense the medication out of the barrel 616, through the needle 618, and into the injection site. As described earlier in this application, the tabs 632 on the casing 606 of the safety syringe 600 are fitted within the indents 634 on the inner side of the plunger cap 602 prior to dispensing of the medication from the syringe 614. The engagement between the tabs 632 and the indents 634 is sufficiently strong to hold the plunger cap 602 and the casing 606 apart while the distal tube 630 is retracted.

After injection of the needle into the injection site and dispensing of the medication, the SSS 100 is in a state of operation shown in FIGS. 8E and 8F, which depict a side view and a side cross-sectional view of the SSS 100. In this state, the inner casing 104 has been proximally retracted to its initial state. At this point, the SSS 100 can be further proximally moved from the injection site, which will cause the distal tube 630 to re-extend over the needle 618. At that point, the cap 608 and syringe cap 636 can be reattached to the safety syringe 600, and a button can be pushed either on the cap 108 or via the display screen 112 that proximally retracts the safety syringe 600 back into its initial, proximal position (albeit, with the safety syringe 600 in a used state), which is shown by FIGS. 8G and 8H.

As seen in FIGS. 8G and 8H, which depict a side view and a side cross-sectional view of the SSS 100 in a fourth state of operation, the used safety syringe 600 has been proximally retracted to its proximal position and the distal tube 630 has been re-extended so as to shield the needle 618. Once the used safety syringe 600 has been securely housed, the microcontroller 110 may begin the timer again based on a preset function (e.g., 12 hours, 24 hours). In this way, the SSS 100 will not permit dispensing of the next (unused) safety syringe 600 until the timer reaches 0:00. At that time, the user can then signal the device (e.g., pushing a button on the cap 108 or on the display screen 112) to dispense the next safety syringe 600.

The following is a description of an exemplary computer or microcontroller that is part of or useable with the safety syringe system 100 described herein. FIG. 9 illustrates an example of a suitable computing and networking environment 900 that may be used to implement various aspects of the present disclosure described herein. As illustrated, the computing and networking environment 900 includes a general purpose computing device 900 (referred to as a computer), although it is contemplated that the networking environment 900 may include other computing systems, such as smart phones, server computers, hand-held or laptop devices, tablet devices, multiprocessor systems, microprocessor-based systems, set top boxes, programmable consumer electronic devices, network PCs, minicomputers, mainframe computers, digital signal processors, state machines, logic circuitries, distributed computing environments that include any of the above computing systems or devices, and the like.

Components of the computer 900 may include various hardware components, such as a processing unit 902, a data storage 904 (e.g., a system memory), and a system bus 906 that couples various system components of the computer 900 to the processing unit 902. The system bus 906 may be any of several types of bus structures including a memory bus or memory controller, a peripheral bus, and a local bus using any of a variety of bus architectures. For example, such architectures may include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

The computer 900 may further include a variety of computer-readable media 908 that includes removable/non-removable media and volatile/nonvolatile media, but excludes transitory propagated signals. Computer-readable media 908 may also include computer storage media and communication media. Computer storage media includes removable/non-removable media and volatile/nonvolatile media implemented in any method or technology for storage of information, such as computer-readable instructions, data structures, program modules or other data, such as RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information/data and which may be accessed by the computer 900. Communication media includes computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. For example, communication media may include wired media such as a wired network or direct-wired connection and wireless media such as acoustic, RF, infrared, and/or other wireless media, or some combination thereof. Computer-readable media may be embodied as a computer program product, such as software stored on computer storage media.

The data storage or system memory 904 includes computer storage media in the form of volatile/nonvolatile memory such as read only memory (ROM) and random access memory (RAM). A basic input/output system (BIOS), containing the basic routines that help to transfer information between elements within the computer 900 (e.g., during start-up) is typically stored in ROM. RAM typically contains data and/or program modules that are immediately accessible to and/or presently being operated on by processing unit 902. For example, in one embodiment, data storage 904 holds an operating system, application programs, and other program modules and program data.

Data storage 904 may also include other removable/non-removable, volatile/nonvolatile computer storage media. For example, data storage 904 may be: a hard disk drive that reads from or writes to non-removable, nonvolatile magnetic media; a magnetic disk drive that reads from or writes to a removable, nonvolatile magnetic disk; and/or an optical disk drive that reads from or writes to a removable, nonvolatile optical disk such as a CD-ROM or other optical media. Other removable/non-removable, volatile/nonvolatile computer storage media may include magnetic tape cassettes, flash memory cards, digital versatile disks, digital video tape, solid state RAM, solid state ROM, and the like. The drives and their associated computer storage media, described above and illustrated in FIG. 9, provide storage of computer-readable instructions, data structures, program modules and other data for the computer 900.

A user may enter commands and information through a user interface 910 or other input devices such as a tablet, electronic digitizer, a microphone, keyboard, and/or pointing device, commonly referred to as mouse, trackball or touch pad. The commands and information may be for setting up the lighting and/or watering schedules, including the specific parameters of each. Other input devices may include a joystick, game pad, satellite dish, scanner, or the like. Additionally, voice inputs, gesture inputs (e.g., via hands or fingers), or other natural user interfaces may also be used with the appropriate input devices, such as a microphone, camera, tablet, touch pad, glove, or other sensor. These and other input devices are often connected to the processing unit 902 through a user interface 910 that is coupled to the system bus 906, but may be connected by other interface and bus structures, such as a parallel port, game port or a universal serial bus (USB). A monitor 912 or other type of display device is also connected to the system bus 906 via an interface, such as a video interface. The monitor 912 may also be integrated with a touch-screen panel or the like.

The computer 900 may operate in a networked or cloud-computing environment using logical connections of a network interface or adapter 914 to one or more remote devices, such as a remote computer. The remote computer may be a personal computer, a server, a router, a network PC, a peer device or other common network node, and typically includes many or all of the elements described above relative to the computer 900. The logical connections depicted in FIG. 9 include one or more local area networks (LAN) and one or more wide area networks (WAN), but may also include other networks. Such networking environments are commonplace in offices, enterprise-wide computer networks, intranets and the Internet.

When used in a networked or cloud-computing environment, the computer 900 may be connected to a public and/or private network through the network interface or adapter 914. In such embodiments, a modem or other means for establishing communications over the network is connected to the system bus 906 via the network interface or adapter 914 or other appropriate mechanism. A wireless networking component including an interface and antenna may be coupled through a suitable device such as an access point or peer computer to a network. In a networked environment, program modules depicted relative to the computer 900, or portions thereof, may be stored in the remote memory storage device.

The foregoing merely illustrates the principles of the invention. Various modifications and alterations to the described embodiments will be apparent to those skilled in the art in view of the teachings herein. It will thus be appreciated that those skilled in the art will be able to devise numerous systems, arrangements and methods which, although not explicitly shown or described herein, embody the principles of the invention and are thus within the spirit and scope of the present invention. From the above description and drawings, it will be understood by those of ordinary skill in the art that the particular embodiments shown and described are for purposes of illustrations only and are not intended to limit the scope of the present invention. References to details of particular embodiments are not intended to limit the scope of the invention.

What is claimed is:

1. A syringe safety system comprising:
at least one safety syringe comprising a first safety syringe comprising a first outer casing configured to enclose a first syringe having a first barrel, a first needle extending distally from the first barrel, and a first plunger insertable within the first barrel to dispense a first drug within the first barrel; and
a dispensing device comprising:
an outer casing comprising a sidewall defining an inner volume, a distal opening, and a proximal opening opposite the distal opening;
a cap configured to couple to and enclose the proximal opening of the outer casing;
a microcontroller configured to be housed within the outer casing and in communication with at least one battery;
a carrier assembly configured to be positioned within the inner volume of the outer casing, the carrier assembly comprising: a carrier clip configured to support the at least one safety syringe around a central axis; a guide tube coupled to the carrier clip and comprising a first track for guiding linear movement of the first safety syringe; a lumen extending through the guide tube; a lead screw positioned within the lumen; a guide tube motor rotatably coupled to the lead screw and in communication with the microcontroller; a carrier motor rotatably coupled to the guide tube and in communication with the microcontroller; a lead nut engaged with the lead screw and comprising a protrusion that is sized and shaped to be received in the first track to guide linear movement of the first safety syringe; and a first plunger coupler including a first plunger support portion and a first protrusion extending outwards from the first plunger support portion, the first protrusion configured to be received within the first track, the first plunger support portion positioned to engage the first plunger of the first syringe, wherein the protrusion of the lead nut is configured to contact the first protrusion of the first plunger coupler in the first track such that linear movement in a distal direction of the lead nut via rotation of the lead screw by the guide tube motor is configured to cause distal advancement of the first plunger support portion and the first safety syringe at least partially out of the distal opening of the outer casing.

2. The syringe safety system of claim 1, wherein:
the at least one safety syringe further comprises a second safety syringe comprising a second outer casing configured to enclose a second syringe having a second barrel, a second needle extending distally from the second barrel, a second plunger insertable within the second barrel to dispense a second drug within the second barrel; and
the guide tube further comprises a second track for guiding linear movement of the second safety syringe, the protrusion of the lead nut being sized and shaped to be received in the second track to guide linear movement of the second safety syringe;
the carrier assembly further comprising a second plunger coupler including a second plunger support portion and a second protrusion extending outwards from the second plunger support portion, the second protrusion configured to be received within the second track, the second plunger support portion positioned to engage the second plunger of the second syringe, wherein the lead nut is configured to be positioned proximally of the second protrusion in the second track such that linear movement in a distal direction of the lead nut via rotation of the lead screw by the guide tube motor is configured to cause distal advancement of the second plunger coupler and the second safety syringe at least partially out of the distal opening of the outer casing.

3. The syringe safety system of claim 2, wherein the microcontroller is programmable to permit actuation of the lead screw in order to distally advance the second safety syringe at least partially out of the distal opening of the outer casing for dispensing of the second drug at a predetermined amount of time after the first safety syringe is distally advanced at least partially out of the distal opening of the outer casing.

4. The syringe safety system of claim 1, wherein, subsequent to distal advancement of the first safety syringe at least partially out of the distal opening of the outer casing, the microcontroller is configured to: proximally retract the first safety syringe into the inner volume of the outer casing via rotation of the lead screw by the guide tube motor; and rotate the carrier assembly within the inner volume of the outer casing such that the second safety syringe is positioned in alignment with the distal opening of the outer casing such that distal advancement of the second safety syringe will position the second safety syringe at least partially out of the distal opening on the distal surface of the outer casing.

5. The syringe safety system of claim 1, wherein the first safety syringe comprises:
the first outer casing comprising a first outer casing distal opening, a first inner surface, and a first track defined on the first inner surface;
a first carrier configured to support the first syringe in a position within the first outer casing;
a first distal tube comprising a first distal end, the first distal tube slidably engaged with the first carrier and configured to be in a first extended state and in a first retracted state, wherein, in the first retracted state, the first needle of the first syringe extends beyond the first distal end and the first outer casing distal opening;
a first biasing member engaged with the first carrier and the first distal tube so as to bias the first distal tube in the first extended state; and
a first ring including a first ring protrusion, the first ring engaged with the first distal tube and the first biasing member, the first ring protrusion positioned at least partially within the first track defined on the first inner surface.

6. The syringe safety system of claim 5, wherein the first safety syringe further comprises:
a first plunger coupler biasing member that is engaged with the first plunger coupler and the first outer casing of the first safety syringe, wherein the first plunger coupler biasing member is configured to release engagement with the first plunger couple upon overcoming a biasing force so as to permit distal advancement of the first plunger coupler relative to the first outer casing.

7. The syringe safety system of claim 6, wherein the biasing force associated with the first plunger coupler biasing member is greater than another biasing force associated with the first biasing member.

8. The syringe safety system of claim 1, wherein the at least one safety syringe comprises the first safety syringe, a second safety syringe, a third safety syringe, and a fourth safety syringe.

9. The syringe safety system of claim 1, further comprising the first drug within the first barrel of the first syringe.

10. The syringe safety system of claim 1, wherein the cap comprises a display device, the microcontroller in communication with the display device.

11. The syringe safety system of claim 1, wherein the first plunger support portion is a cap.

12. The syringe safety system of claim 1, wherein the dispensing device further includes an inner casing that is fitted at least partially within the inner volume of the outer casing, the inner casing being slidable relative to the outer casing.

13. The syringe safety system of claim 12, wherein distal advancement of the first safety syringe at least partially out of the distal opening of the outer casing causes distal advancement of the inner casing.

14. The syringe safety system of claim 13, wherein the first safety syringe further comprises a ring that contacts the inner casing and causes the inner casing to advance distally when the first safety syringe is advanced distally.

15. (canceled)

16. A system, comprising:
a safety syringe system comprising a dispensing device and at least one safety syringe supported within the dispensing device and configured to be dispensed from the dispensing device, the dispensing device comprising a carrier assembly supporting the at least one safety syringe and comprising at least one motor, a casing comprising a distal opening and enclosing the carrier assembly and the at least one safety syringe, and a computing device in communication with the at least one motor and having one or more processors and a memory storing instructions that, as a result of being executed by the one or more processors, cause the system to perform the operations including:
actuating a first motor of the at least one motor to dispense a first safety syringe of the at least one safety syringe at least partially out of the distal opening of the casing.

17. The system of claim 16, wherein actuating the first motor is permitted only at a predetermined time.

18. The system of claim 17, wherein the predetermined time is when a dosage of a medicament within the first safety syringe is due for a user.

19. The system of claim 16, wherein the dispensing device further comprises a display screen in communication with the one or more processors.

20. (canceled)

21. A syringe safety system comprising:
a dispensing device configured to enclose and dispense one or more syringes, the dispensing device comprising:
an outer casing comprising a sidewall defining an inner volume, a distal opening, and a proximal opening opposite the distal opening;
a cap configured to couple to and enclose the proximal opening of the outer casing;
a microcontroller configured to be housed within the outer casing and in communication with at least one battery;
a carrier assembly configured to support the one or more syringes within the inner volume of the outer casing, the carrier assembly comprising a drive system in communication with the microcontroller, the drive system configured to provide movement of a first syringe of the one or more syringes from within the inner volume of the outer casing to at least partially out of the inner volume of the outer casing.

22-25. (canceled)

26. The system of claim 21, wherein the microcontroller restricts operation of the drive system until a pre-programmed release time.

27-30. (canceled)
